(19) United States
(12) Patent Application Publication
(10) Pub. No.: US 2018/0271721 A1
(43) Pub. Date: Sep. 27, 2018

(54) Combination Catamenial Tampon and Wipe

(71) Applicant: Caren A. Evanosich, Bensalem, PA (US)

(72) Inventor: Caren A. Evanosich, Bensalem, PA (US)

(21) Appl. No.: 15/469,760

(22) Filed: Mar. 27, 2017

(51) Int. Cl. A61F 13/551; A61F 13/26; B65D 75/58

(52) U.S. Cl. CPC A61F 13/5519; B65D 75/5855; A61F 13/55185; A61F 13/26

(57) ABSTRACT

A catamenial tampon product combines a catamenial tampon and a personal hygiene cleansing wipe in one convenient package. The personal hygiene cleansing wipe is contained in an individual sealed package. The catamenial tampon product includes a pouch that contains the catamenial tampon. In some embodiments, the personal hygiene cleansing wipe is also contained within the pouch. Some embodiments of the catamenial tampon product have an opening that is covered by an adhesive panel or a closure flap.

### BACKGROUND OF THE INVENTION

It is known in the art to use catamenial tampons to prevent the soiling of undergarments during menstruation. Various tampon and sanitary napkin products have been proposed in the prior art; however, the need remains for a catamenial tampon product that combines a catamenial tampon and a personal hygiene cleansing wipe in one convenient package.

### SUMMARY OF THE INVENTION

The present invention provides a catamenial tampon product that combines a catamenial tampon and a personal hygiene cleansing wipe in one convenient package.

The present invention comprises an improved article consisting of the combination of a catamenial tampon, such as disclosed, for example, in U.S. Pat. Nos. 3,749,094, 3,900,030, 4,018,225, 5,795,346, 5,807,372, 6,095,998, 6,595,974, 6,773,423, 6,887,226, 8,353,888, 8,585,668, each incorporated herein in their entirety by reference, and a pre-moistened wipe, such as disclosed, for example, in U.S. Pat. Nos. 5,629,081, 6,207,596, 6,641,826, 6,916,480, 8,603,297, each incorporated herein in their entirety by reference, packaged together. Preferably, both the tampon and the pre-moistened wipe are flushable and biodegradable. In one presently preferred embodiment, a single tampon and a single wipe are packaged together. In another embodiment, a single tampon and a plurality of wipes are package together. Preferably, the wipe is separately packaged and sealed within the packaging for the tampon. Alternatively, the wipe is separately packaged, and attached to the exterior of the tampon packaging, such as with a suitable adhesive material, such as a hot-melt adhesive. The present invention advantageously permits a user to have immediate access to a wipe for cleaning herself after removing a soiled tampon.

Accordingly, it is an aspect of the present invention to provide a feminine hygiene kit comprising:

a catamenial tampon;

a tampon applicator, the catamenial tampon being contained in the tampon applicator;

a cleansing, moist wipe or towelette contained in an individual sealed package; and

a pouch containing the tampon applicator having the catamenial tampon therein and the sealed package containing the towlette, the pouch being sealed so as to maintain the tampon and the applicator in a sanitary condition, the pouch having contents comprising the tampon applicator having the catamenial tampon therein and the sealed package containing the towlette, the pouch being openable by a user to access the tampon applicator having the catamenial tampon therein and the sealed package containing the towlette when the user elects to open the pouch and access the contents of the pouch.

It is another aspect of the present invention to provide a feminine hygiene kit as set forth above, wherein the sealed package containing the towlette is adhesively secured to an interior surface of the pouch.

It is yet another aspect of the present invention to provide a feminine hygiene kit incorporating any of the above-described aspects of the present invention, wherein the pouch has an opening and closure means that can maintain the pouch opening in a closed condition until the pouch is opened by the user.

It is yet another aspect of the present invention to provide a feminine hygiene kit incorporating any of the above-described aspects of the present invention, wherein the closure means is an adhesive panel that sealingly covers the pouch opening.

It is yet another aspect of the present invention to provide a feminine hygiene kit incorporating any of the above-described aspects of the present invention, wherein the sealed package containing the towlette is adhesively secured to an interior surface of the adhesive panel.

It is yet another aspect of the present invention to provide a feminine hygiene kit incorporating any of the above-described aspects of the present invention, wherein the closure means is a closure flap, the pouch opening being defined at least in part by an edge that is part of an edge portion of the pouch that at least in part surrounds the opening, the closure flap being movable between a closed position and an open position, the closure flap being secured in the closed position by one of an adhesive attachment and a frangible attachment between the flap and the edge portion of the pouch.

It is yet another aspect of the present invention to provide a feminine hygiene kit incorporating any of the above-described aspects of the present invention, wherein the sealed package containing the towlette is adhesively secured to an interior surface of the closure flap.

It is yet another aspect of the present invention to provide a feminine hygiene kit comprising:

a catamenial tampon;

a tampon applicator, the catamenial tampon being contained in the tampon applicator;

a pouch containing the tampon applicator having the catamenial tampon therein, the pouch being sealed so as to maintain the tampon and the applicator in a sanitary condition, the pouch having contents comprising the tampon applicator having the catamenial tampon therein, the pouch being openable by a user to access the tampon applicator having the catamenial tampon therein when the user elects to open the pouch and access the contents of the pouch, the pouch having an exterior; and

a cleansing, moist wipe or towelette contained in a sealed individual package adhesively secured to the exterior of the pouch.

It is yet another aspect of the present invention to provide a feminine hygiene kit incorporating any of the above-described aspects of the present invention, wherein the sealed package is adapted to be openable by the user to access the towelette contained in the sealed package when the user elects to do so.

It is yet another aspect of the present invention to provide a feminine hygiene kit incorporating any of the above-described aspects of the present invention, wherein the pouch has an opening and closure means that can maintain the pouch opening in a closed condition until the pouch is opened by the user.

It is yet another aspect of the present invention to provide a feminine hygiene kit incorporating any of the above-described aspects of the present invention, wherein the sealed package containing the towlette is adhesively secured to an exterior surface of the closure means.

It is yet another aspect of the present invention to provide a feminine hygiene kit incorporating any of the above-described aspects of the present invention, wherein the closure means is an adhesive panel that sealingly covers the pouch opening.

It is yet another aspect of the present invention to provide a feminine hygiene kit incorporating any of the above-described aspects of the present invention, wherein the sealed package containing the towlette is adhesively secured to an exterior surface of the adhesive panel.

It is yet another aspect of the present invention to provide a feminine hygiene kit incorporating any of the above-described aspects of the present invention, wherein the closure means is a closure flap, the pouch opening being defined at least in part by an edge that is part of an edge portion of the pouch that at least in part surrounds the opening, the closure flap being movable between a closed position and an open position, the closure flap being secured in the closed position by one of an adhesive attachment and a frangible attachment between the flap and the edge portion of the pouch.

It is yet another aspect of the present invention to provide a feminine hygiene kit incorporating any of the above-described aspects of the present invention, wherein the sealed package containing the towlette is adhesively secured to an exterior surface of the closure flap.

It is yet another aspect of the present invention to provide a feminine hygiene kit incorporating any of the above-described aspects of the present invention, wherein the pouch is adapted to be tearable to allow the pouch to be opened by the user.

It is yet another aspect of the present invention to provide a feminine hygiene kit incorporating any of the above-described aspects of the present invention, wherein the pouch has an opening, the pouch opening being defined at least in part by an edge that is part of an edge portion of the pouch that at least in part surrounds the opening, the opening being sealingly secured in a closed configuration by portions of the edge portion of the pouch being sealingly attached together in a sealing attachment, and wherein the pouch can be opened by pulling apart the portions of the edge portion or by breaking open the pouch proximate the edge portion.

These and other aspects and advantages of the present invention will be further elucidated by the following Detailed Description, drawing figures, and Claims.

### BRIEF DESCRIPTION OF THE DRAWING FIGURES

The attached drawings show embodiments of the invention in the several views.

FIGS. 1 and 2 show views of a tampon and tampon applicator assembly.

FIGS. 3-6 show views of the wet personal hygiene wipe or towelette and the sealed individual towelette package.

FIGS. 7-9 show a first embodiment of the pouch or wrapper of the present invention containing the tampon and applicator assembly and the sealed wipe package that employs an adhesive panel as the closure for the pouch opening.

FIGS. 10-11 show a second embodiment of the pouch or wrapper of the present invention containing the tampon and applicator assembly inside with the sealed wipe package adhesively attached to the exterior of the pouch, and this embodiment employs an adhesive panel as the closure for the pouch opening.

FIGS. 12-13 show a third embodiment of the pouch or wrapper of the present invention containing the tampon and applicator assembly and the sealed wipe package that employs a closure panel with a frangible attachment to the edges of the opening as the closure for the pouch opening.

FIGS. 14-15 show a fourth embodiment of the pouch or wrapper of the present invention containing the tampon and applicator assembly inside with the sealed wipe package adhesively attached to the exterior of the pouch, and this embodiment employs a closure panel with a frangible attachment to the edges of the opening as the closure for the pouch opening.

FIGS. 16-17 show a fifth embodiment of the pouch or wrapper of the present invention containing the tampon and applicator assembly and the sealed wipe package that employs a closure panel as the closure for the pouch opening that is secured in the closed position by an adhesive strip.

FIGS. 18-19 show a sixth embodiment of the pouch or wrapper of the present invention containing the tampon and applicator assembly and the sealed wipe package that employs a closure panel as the closure for the pouch opening that is secured in the closed position by an overlying adhesive panel.

FIGS. 20-21 show a seventh embodiment of the pouch or wrapper of the present invention containing the tampon and applicator assembly and the sealed wipe package that can be opened by pulling apart the sealed edges of one end of the pouch or wrapper.

### DETAILED DESCRIPTION

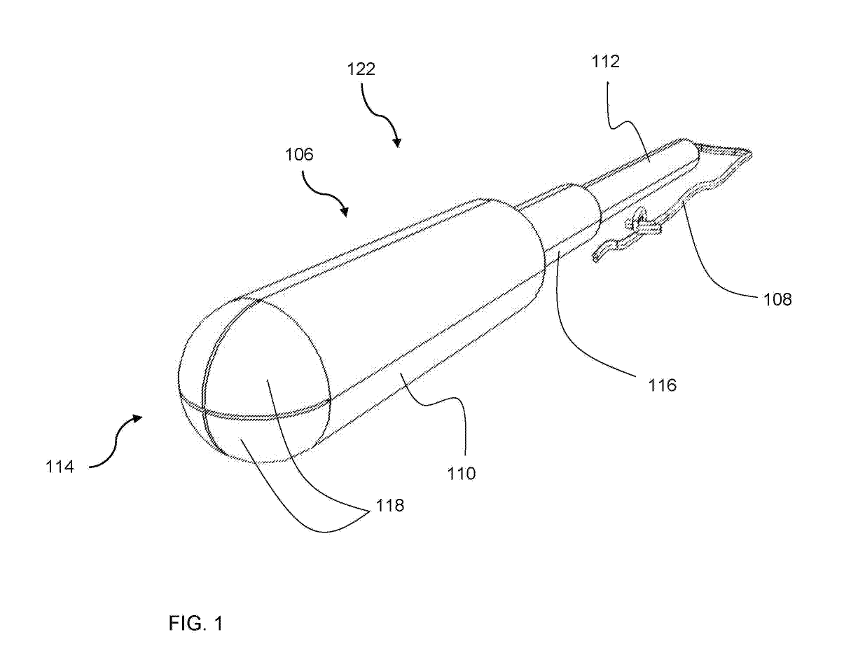

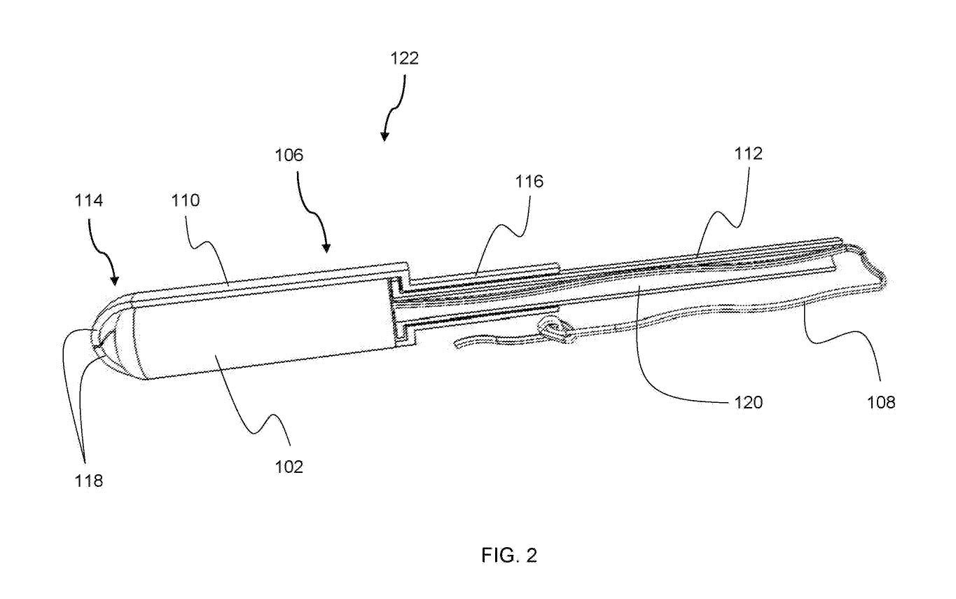

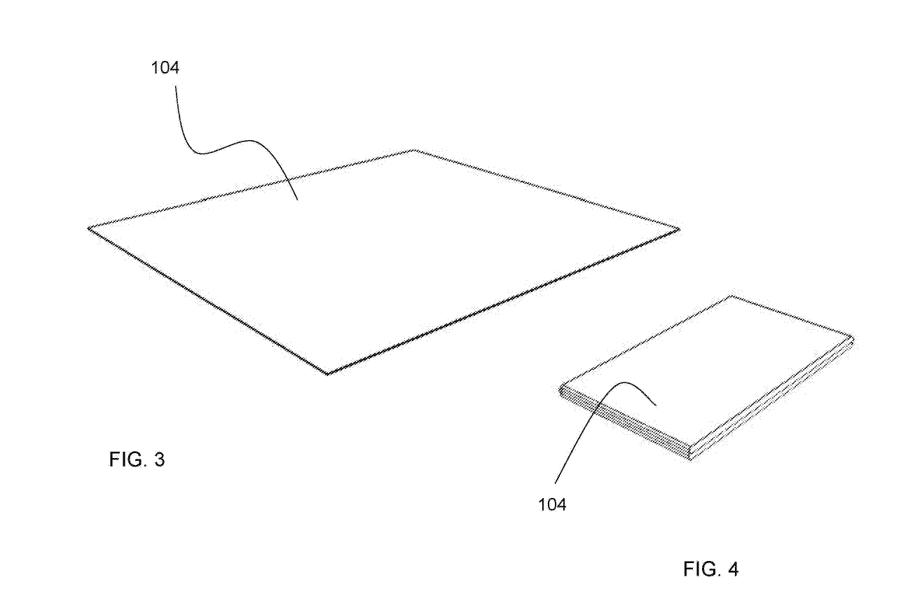

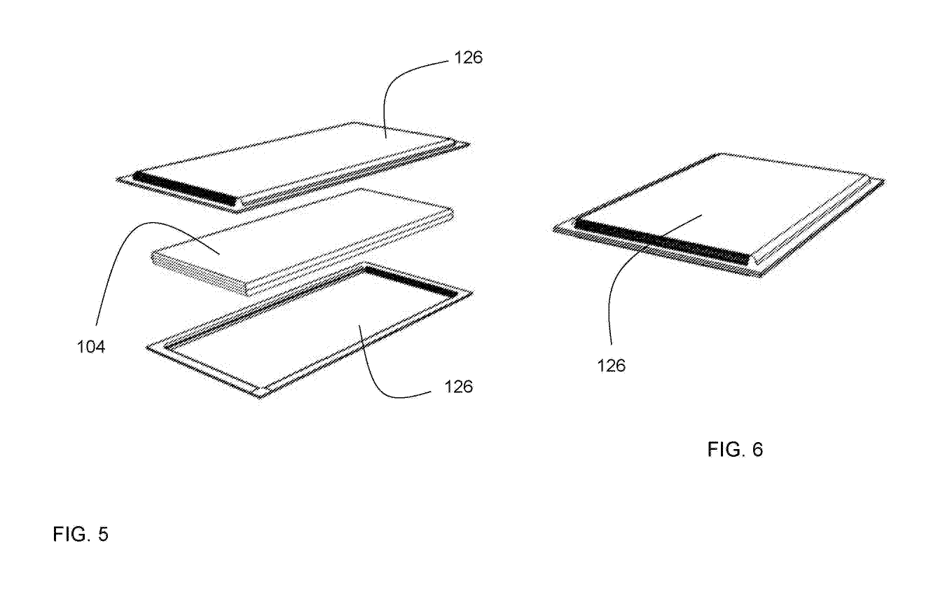

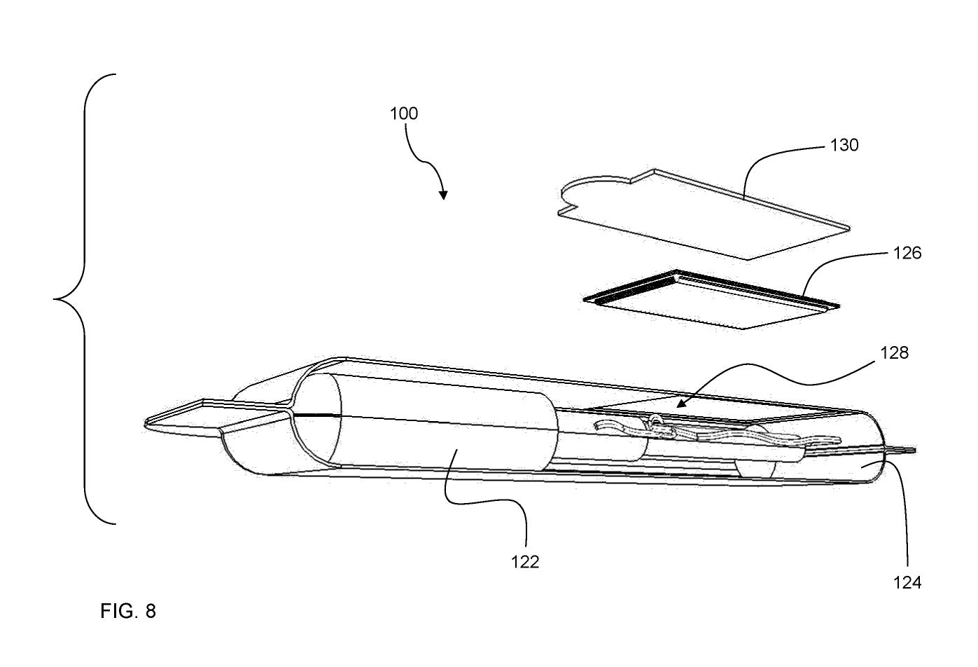

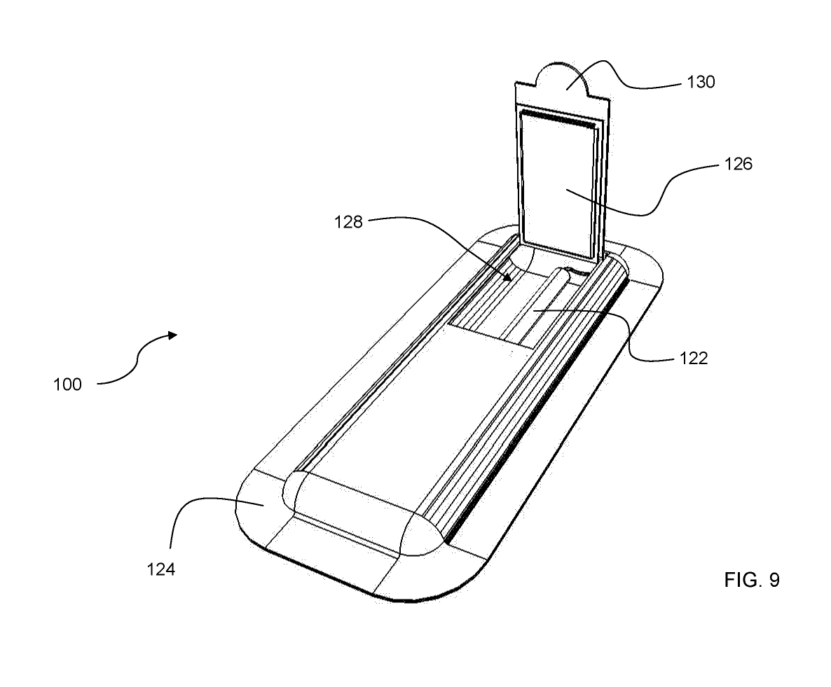

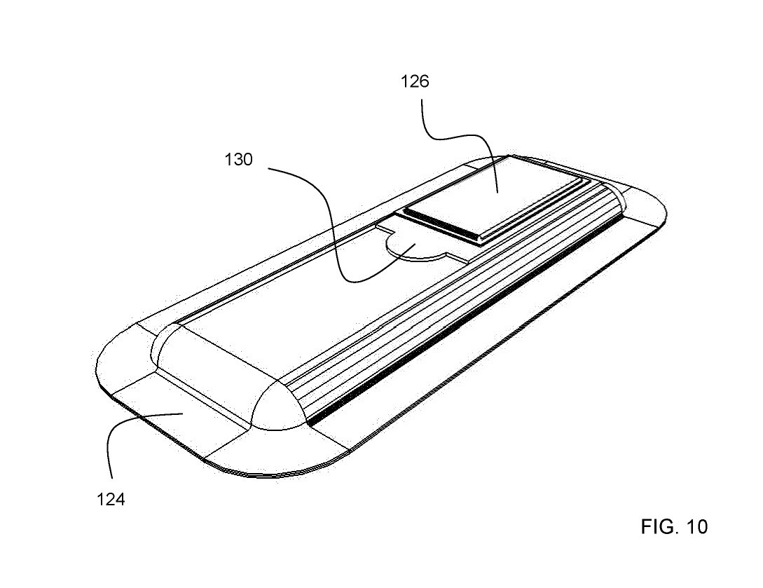

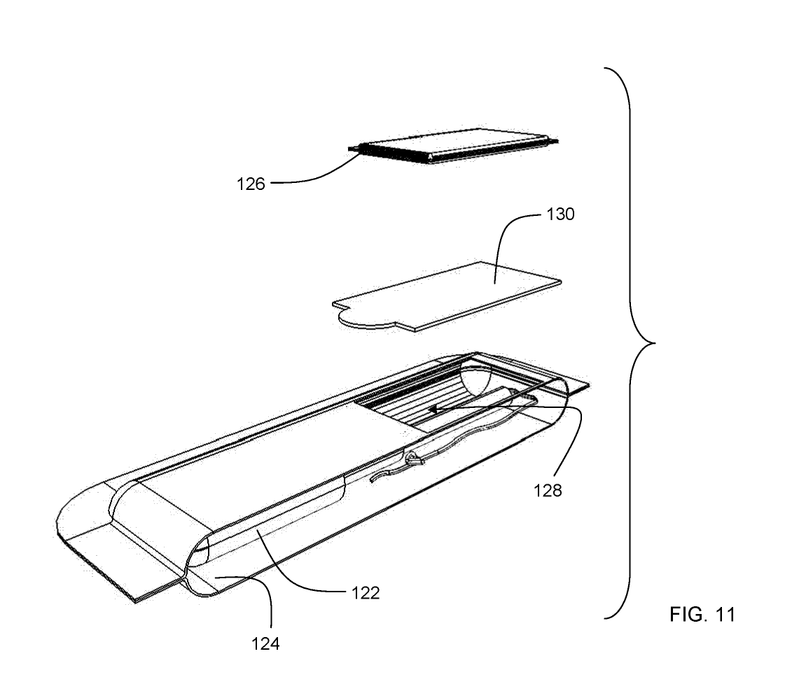

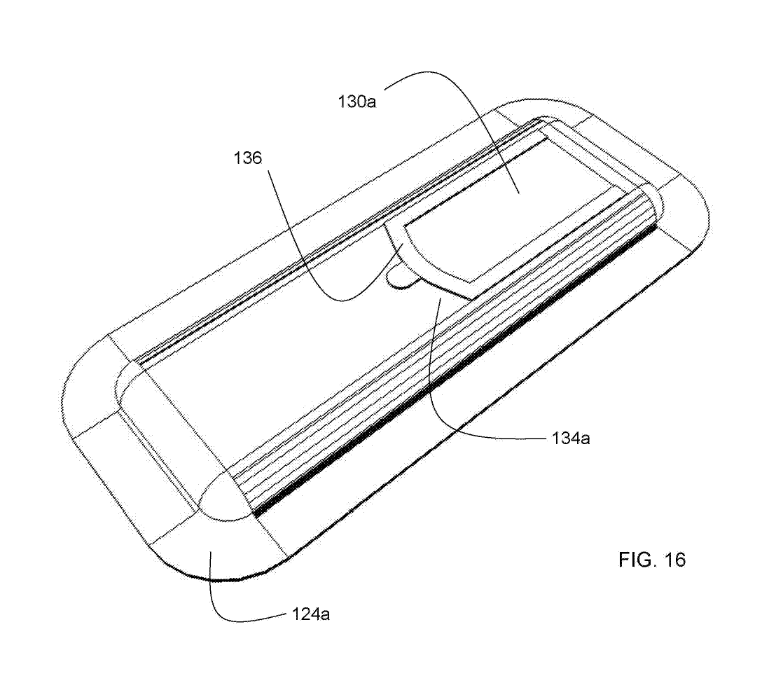

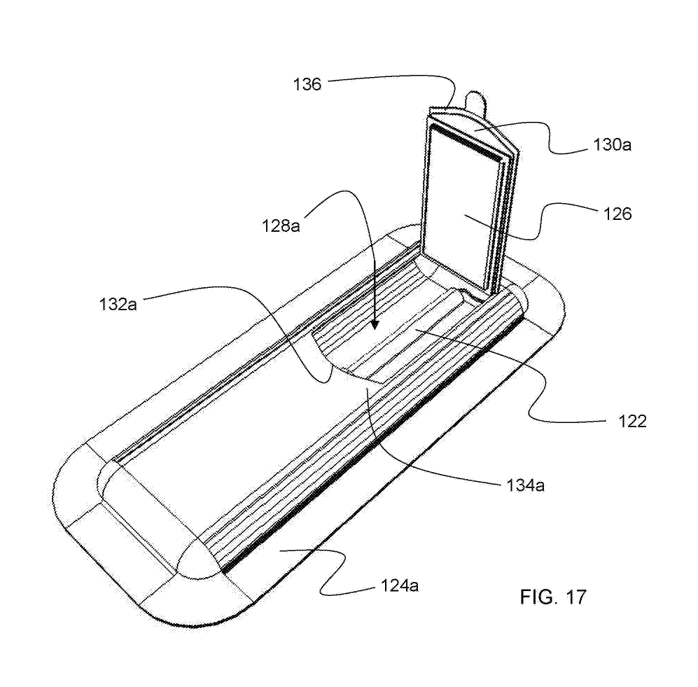

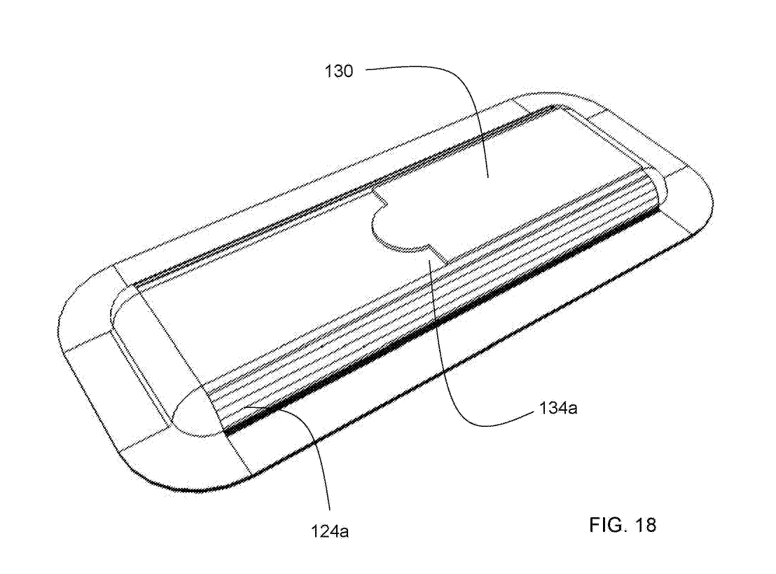

Referring to FIGS. 1-17, the present invention is directed to a feminine hygiene kit 100 that includes a catamenial tampon 102 and a cleansing, moist wipe or towelette 104 contained in a single package. The kit may also include a tampon applicator 106. The catamenial tampon 102 would then be contained in the tampon applicator 106. The catamenial tampon 102 includes a pull cord or string 108 to facilitate removal of the tampon 102 after use. The tampon applicator 106 includes a barrel 110 and a plunger 112. The barrel 110 has smooth and rounded tip 114 for easy insertion into the vaginal opening. The tampon 102 is contained in the barrel 110 of the applicator. The barrel 110 has a reduced diameter gripping portion 116 that allows the barrel to be gripped with the thumb and the middle finger while the index finger is used to push the plunger further into the barrel 110 in order to expel the tampon from the barrel 110 and into place in the vaginal canal. Perforations or cuts in the tip 114 of the barrel 110 allow the formation of petals 118 in the flexible material of the tip 114, which is usually made of plastic or polymer material, that spread apart to allow the tampon 102 to be expelled from the barrel 110 of the applicator as the plunger 112 of the applicator is pushed further into the barrel 110.

The tampon 102 is made of absorbent material, which is usually cotton or cotton-like, that can absorb the menstrual flow and expands to occlude the vaginal canal and prevent the leakage of menstrual fluids from the wearer's body. The tampon 102 is provided with a string or cord 108 that remains at least in part outside the body after the tampon 102 is positioned within the vaginal canal. The string 108 can be grasped to remove the tampon 102 from the vaginal canal after it has been in use for a time period that preferably does not exceed the recommended time period for a single tampon to be in continuous use, which is usually in the range of 4 to 8 hours. The tampon is then replaced with a fresh tampon if needed to continue prevention of menstrual fluid leakage. The plunger 112 has a hollow bore 120 that allows the string 108 to extend through the plunger 112 with a portion of the string 108 remaining outside the applicator 106 before, during, and after the placement of the tampon 102 in the vaginal canal. After the tampon 102 is positioned within the vaginal canal, the applicator 106 can be removed and discarded while leaving the tampon 102 and the string 108 properly positioned relative to the wearer's body.

The tampon and applicator assembly 122 is contained in an enclosed wrapper 124 designed to prevent contamination of the assembly 122 and maintain the sanitary and/or hygienic condition of the assembly 122 until the assembly 122 is needed for use. At that time, the assembly 122 is removed from the wrapper 124 for use.

The cleansing, moist wipe or towelette 104 is contained in an individual sealed package or wrapper 126 designed to prevent contamination of the wipe 104 and maintain the sanitary and/or hygienic condition of the wipe 104 until the wipe 104 is needed for use. At that time, the wipe 104 is removed from the wrapper 126 for use. The wrapper 126 also acts to prevent the evaporation of moisture and the drying of the wipe 104. The removal of the wipe 104 from the wrapper 126 can be accomplished by tearing open the wrapper 126. The wrapper 126 may be provided with a small starter cut within the sealed border of the wrapper 126, at the outside edge of the sealed border, to aid in the opening of the wrapper 126. Alternatively, the wrapper 126 may be provided with a string-type tearing aid or with an opening having an adhesive closure. Preferably, the wipe 104 is moist and is also impregnated with a cleansing, sanitizing, and/or antiseptic agent or composition. The wipe 104 is intended to be used to cleanse the genital area of the person that will be using the tampon 102, before and/or after the application of the tampon 102, to reduce the likelihood of infections that can result in deleterious health effects and to impart a feeling of cleanliness and freshness to the wearer of the tampon 102. The towelette 104 is preferably of the flushable variety.

In one embodiment, the pouch or wrapper 124 contains both the tampon applicator 106 having the catamenial tampon 102 therein and the sealed package containing the towlette inside the pouch or wrapper 124. The pouch 124 is openable by a user, which likely is the person intending to wear the tampon, to access the tampon applicator 106 having the catamenial tampon 102 therein and the sealed package 126 containing the towlette or wipe 104 when the user elects to open the pouch 124 and access the contents of the pouch 124. Preferably, the wrapper or pouch 124 is flexible and is made of a flexible material such as paper, polymeric material, plastics, and combinations thereof.

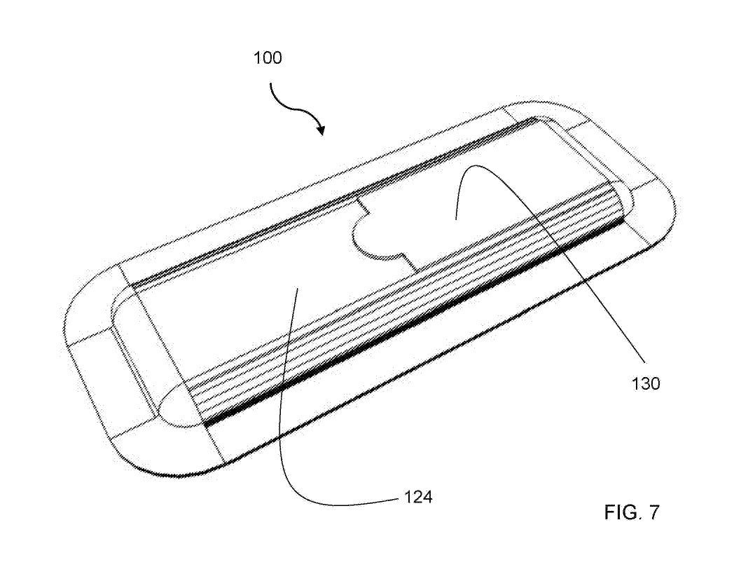

In this embodiment (FIGS. 7-9), the sealed package 126 containing the towlette 104 is adhesively secured to an interior surface of the pouch 124. The pouch 124 may be tearable to allow opening of the pouch 124 to access its contents. In the illustrated embodiment, the pouch is provided with an opening 128 to allow access to the tampon 102 and the towlette 104 and closure means 130 that can maintain the pouch opening 128 in a closed condition until the pouch is opened by the user. In the illustrated embodiment, the closure means is an adhesive panel 130 that sealingly covers the pouch opening 128.

Preferably, the sealed package 126 containing the towlette 104 is adhesively secured to the interior surface of the adhesive panel 130 such that, as the adhesive panel 130 is opened, the package 126 containing the towelette 104 is simultaneously extracted from the wrapper or pouch 124 and presented for ready access by the user.

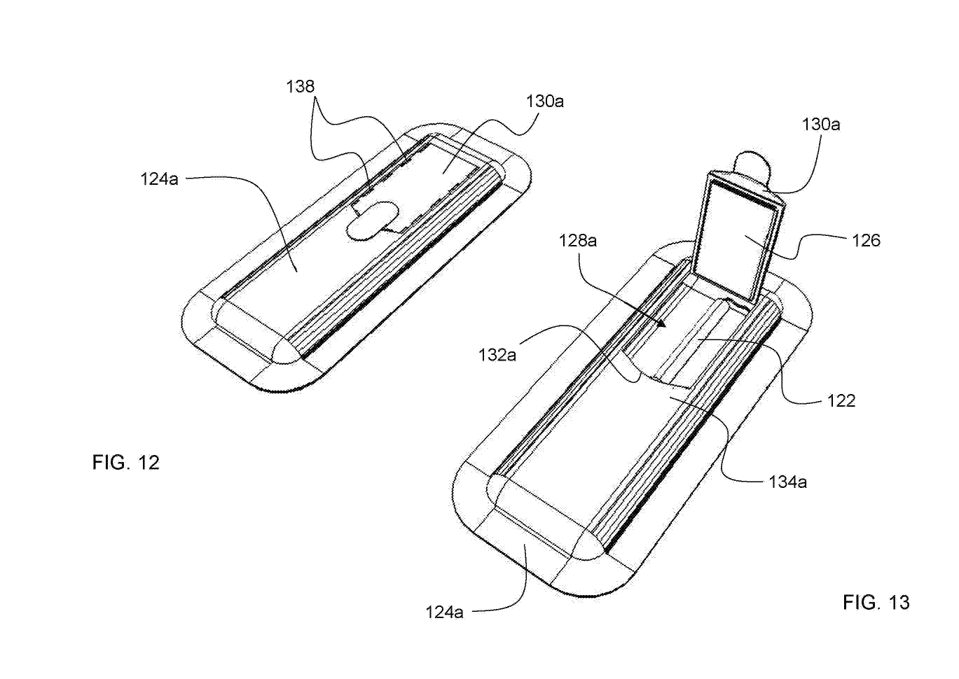

Alternatively, the closure means can be a closure flap 130a as shown in FIGS. 12, 13, 16, and 17. The pouch opening 128a is defined at least in part by an edge 132a that is part of an edge portion 134a of the pouch 124a that at least in part surrounds the opening 128a. The closure flap 130a is movable between a closed position and an open position. The closure flap 130a is secured in the closed position by an adhesive attachment, for example using an adhesive strip 136, or a frangible attachment 138, or both, between the flap 130a and the edge portion 134a of the pouch 124a. The sealed package 126 containing the towlette 104 may be adhesively secured to the interior surface of the closure flap 130a such that, as the closure flap 130a is opened, the package 126 containing the towelette 104 is simultaneously extracted from the wrapper or pouch 124a and presented for ready access by the user. The frangible attachment may be formed by a line of perforations, or preferably embossed indentations, distributed along the line of demarcation between the closure panel and the opening of the pouch 124a.

As yet another alternative embodiment, the cleansing, moist wipe or towelette 104 may be contained in a sealed individual package 126 that is adhesively secured to the exterior of the pouch 124 as shown in FIGS. 10, 11, 14, and 15. The sealed package 126 is adapted to be openable by the user to access the towelette 104 contained in the sealed package 126 when the user elects to do so.

As with the previously described embodiments, the pouch 124 has an opening 128 and closure means 130 that can maintain the pouch opening 128 in a closed condition until the pouch 124 is opened by the user. Preferably, the sealed package 126 containing the towlette 104 is adhesively secured to an exterior surface of the closure means 130. In the illustrated embodiment, the closure means 130 is an adhesive panel 130 that sealingly covers the pouch opening 128. In the illustrated embodiment, the sealed package 126 containing the towlette 104 is adhesively secured to an exterior surface of the adhesive panel 130 such that the package 126 can be grasped by the user to pull the adhesive panel 130 to the open position or configuration. In this way, the package 126 affords the user a better purchase for opening the adhesive panel 130.

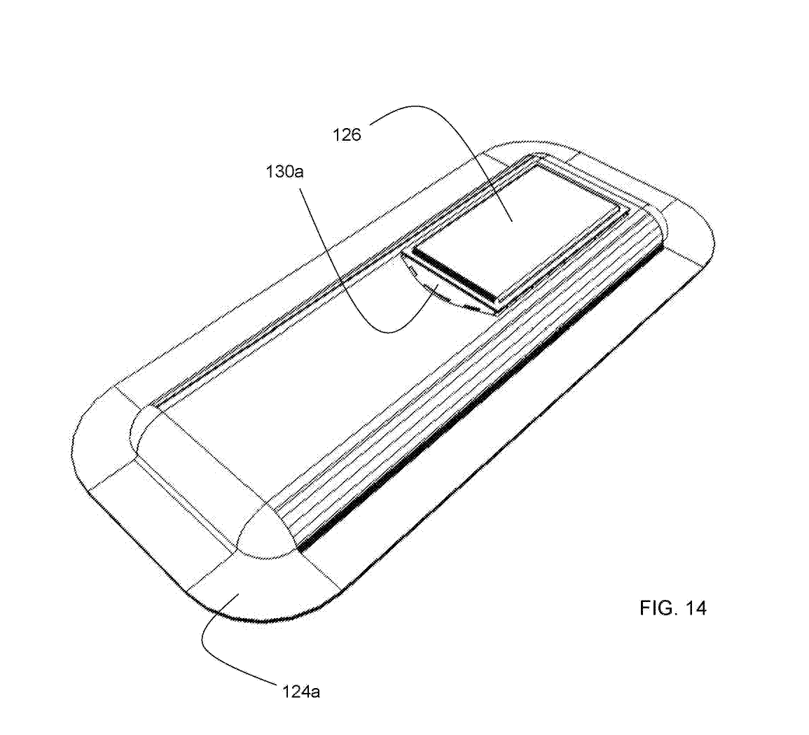

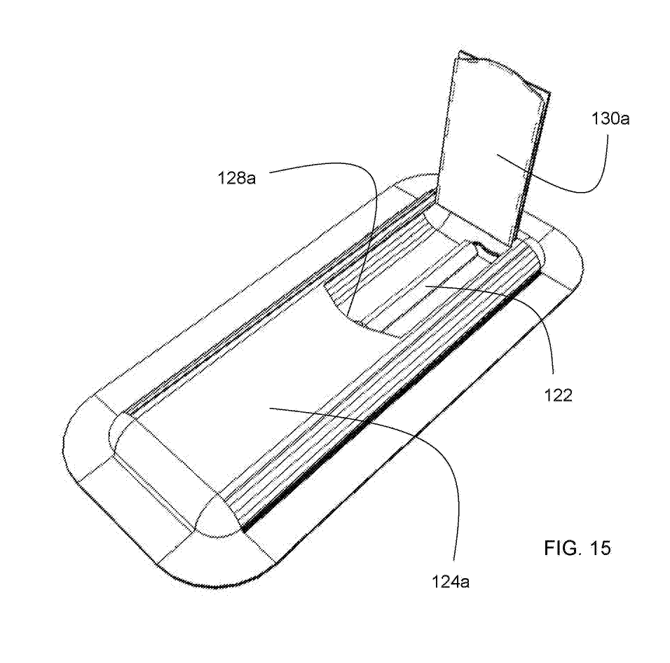

Alternatively (FIGS. 14 and 15), the closure means can be a closure flap 130a. The pouch opening 128a is defined at least in part by an edge 132a that is part of an edge portion 134a of the pouch 124a that at least in part surrounds the opening 128a. The closure flap 130a is movable between a closed position and an open position. The closure flap 130a is secured in the closed position by an adhesive attachment 136 or a frangible attachment 138, or both, between the flap 130a and the edge portion 134a of the pouch 124a. The sealed package 126 containing the towlette 104 may be adhesively secured to the exterior surface of the closure flap 130a such that the package 126 can be grasped by the user to pull the closure flap 130a to the open position or configuration. In this way, the package 126 affords the user a better purchase for opening the closure flap 130a.

As yet alternative, the pouch 124 can be adapted to be tearable to allow the pouch to be opened by the user.

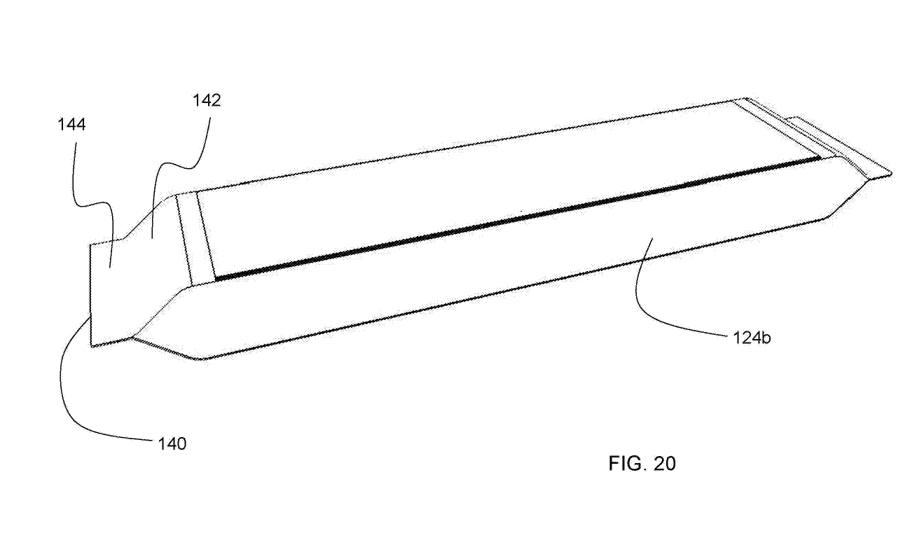

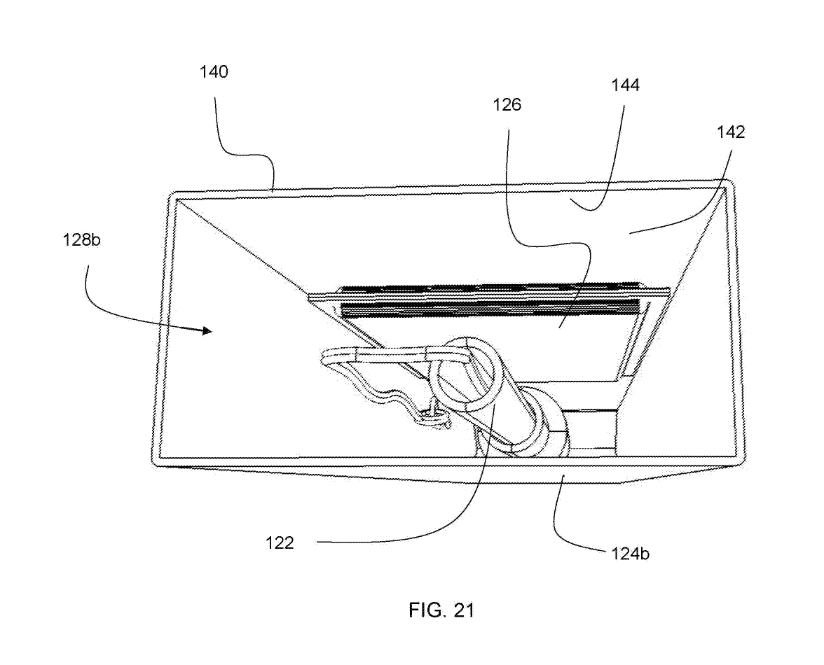

As yet another alternative (shown in FIGS. 20 and 21), the pouch 124b has an opening 128b that is defined at least in part by an edge 140 that is part of an edge portion 142 of the pouch 124b that at least in part surrounds the opening 128b. The opening 128b is sealingly secured in a closed configuration by portions 144 of the edge portion 142 of the pouch 124b that are sealingly attached together in a sealing attachment. The pouch 124b can be opened by pulling apart the portions 144 of the edge portion 142 or by breaking open the pouch 124b proximate the edge portion 142.

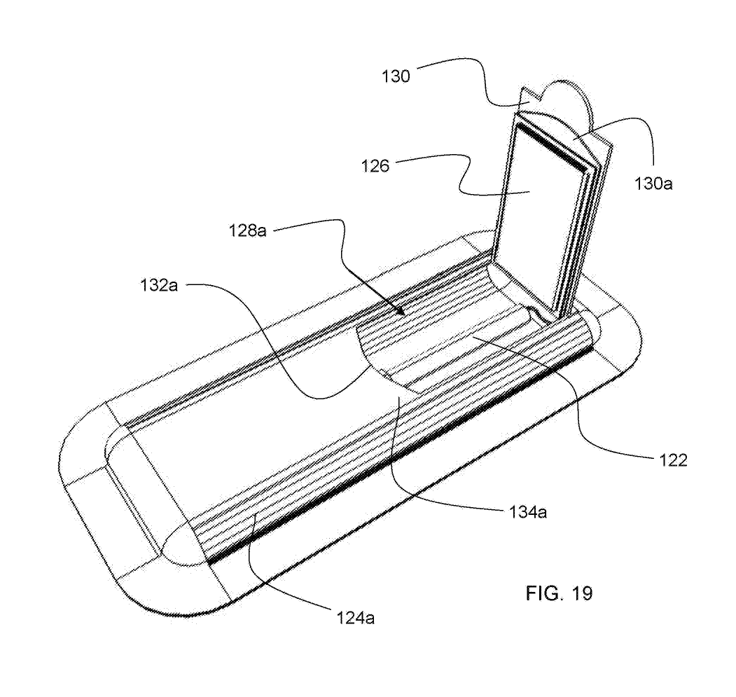

As yet another alternative (FIGS. 18 and 19), the pouch 124a may employ the closure flap 130a over the opening 128a and an adhesive panel 130 attached over the exterior of the closure flap 130a and spanning adjacent portions of the closure flap 130a and the opening edge portion 134a of the pouch 124a.

It should be understood that the present invention is not limited to the specific embodiments described above, but includes any and all variations or modifications within the spirit and scope of the present invention as defined in the appended claims.

1. A feminine hygiene kit comprising:
a catamenial tampon;
a tampon applicator, said catamenial tampon being contained in said tampon applicator;
a cleansing, moist wipe or towelette contained in an individual sealed package; and
a pouch containing said tampon applicator having said catamenial tampon therein and said sealed package containing said towelette, said pouch being sealed so as to maintain said tampon and said applicator in a sanitary condition, said pouch having contents comprising said tampon applicator having said catamenial tampon therein and said sealed package containing said towelette, said pouch being openable by a user to access said tampon applicator having said catamenial tampon therein and said sealed package containing said towelette when the user elects to open said pouch and access said contents of said pouch.

2. The feminine hygiene kit according to claim 1, wherein said sealed package containing said towelette is adhesively secured to an interior surface of said pouch.

3. The feminine hygiene kit according to claim 1, wherein said sealed package is adapted to be openable by the user to access said towelette contained in the sealed package when the user elects to do so.

4. The feminine hygiene kit according to claim 1, wherein said pouch has an opening and closure means configured to maintain the pouch opening in a closed condition until the pouch is opened by the user.

5. The feminine hygiene kit according to claim 4, wherein said closure means is an adhesive panel that sealingly covers said pouch opening.

6. The feminine hygiene kit according to claim 5, wherein said sealed package containing said towelette is adhesively secured to an interior surface of said adhesive panel.

7. The feminine hygiene kit according to claim 4, wherein said closure means is a closure flap, said pouch opening being defined at least in part by an edge that is part of an edge portion of said pouch that at least in part surrounds said opening, said closure flap being movable between a closed position and an open position, said closure flap being secured in the closed position by one or both of an adhesive attachment and a frangible attachment between the flap and the edge portion of said pouch.

8. The feminine hygiene kit according to claim 7, wherein said sealed package containing said towelette is adhesively secured to an interior surface of said closure flap.

9. The feminine hygiene kit according to claim 1, wherein said pouch is adapted to be tearable to allow said pouch to be opened by the user.

10. The feminine hygiene kit according to claim 1, wherein said pouch has an opening, said pouch opening being defined at least in part by an edge that is part of an edge portion of said pouch that at least in part surrounds said opening, said opening being sealingly secured in a closed configuration by portions of the edge portion of the pouch being sealingly attached together in a sealing attachment, and wherein said pouch is openable by pulling apart said portions of the edge portion or by breaking open said pouch proximate said edge portion.

11. A feminine hygiene kit comprising:
a catamenial tampon;
a tampon applicator, said catamenial tampon being contained in said tampon applicator;
a pouch containing said tampon applicator having said catamenial tampon therein, said pouch being sealed so as to maintain said tampon and said applicator in a sanitary condition, said pouch having contents comprising said tampon applicator having said catamenial tampon therein, said pouch being openable by a user to access said tampon applicator having said catamenial tampon therein when the user elects to open said pouch and access said contents of said pouch, the pouch having an exterior; and
a cleansing, moist wipe or towelette contained in a sealed individual package adhesively secured to the exterior of said pouch,
wherein said pouch has an opening and closure means configured to maintain the pouch opening in a closed condition until the pouch is opened by the user, and
wherein said closure means is a closure flap, said pouch opening being defined at least in part by an edge that is part of an edge portion of said pouch that at least in part surrounds said opening, said closure flap being movable between a closed position and an open position, said closure flap being secured in the closed position by one of a frangible attachment and a combination of an adhesive attachment and a frangible attachment between the flap and the edge portion of said pouch.

12. The feminine hygiene kit according to claim 11, wherein said sealed package is adapted to be openable by the user to access said towelette contained in the sealed package when the user elects to do so.

13. (canceled)

14. The feminine hygiene kit according to claim 11, wherein said sealed package containing said towelette is adhesively secured to an exterior surface of said closure means.

15. The feminine hygiene kit according to claim 11, wherein said closure means is an adhesive panel that sealingly covers said pouch opening.

16. The feminine hygiene kit according to claim 15, wherein said sealed package containing said towelette is adhesively secured to an exterior surface of said adhesive panel.

17. (canceled)

18. The feminine hygiene kit according to claim 11, wherein said sealed package containing said towelette is adhesively secured to an exterior surface of said closure flap.

19. (canceled)

20. A feminine hygiene kit comprising:
a catamenial tampon;
a tampon applicator, said catamenial tampon being contained in said tampon applicator;
a pouch containing said tampon applicator having said catamenial tampon therein, said pouch being sealed so as to maintain said tampon and said applicator in a sanitary condition, said pouch having contents comprising said tampon applicator having said catamenial tampon therein, said pouch being openable by a user to access said tampon applicator having said catamenial tampon therein when the user elects to open said pouch and access said contents of said pouch, the pouch having an exterior; and
a cleansing, moist wipe or towelette contained in a sealed individual package adhesively secured to the exterior of said pouch, wherein said pouch has an opening, said pouch opening being defined at least in part by an edge that is part of an edge portion of said pouch that at least in part surrounds said opening, said opening being sealingly secured in a closed configuration by portions of the edge portion of the pouch being sealingly attached together in a sealing attachment, and wherein said pouch is openable by pulling apart said portions of the edge portion or by breaking open said pouch proximate said edge portion.

21. The feminine hygiene kit according to claim 7, further comprising an adhesive panel attached over the exterior of said closure flap and spanning adjacent portions of said closure flap and said edge portion of said pouch.

22. The feminine hygiene kit according to claim 11, further comprising an adhesive panel attached over the exterior of said closure flap and spanning adjacent portions of said closure flap and said edge portion of said pouch.

23. The feminine hygiene kit according to claim 7, wherein said closure flap is secured in the closed position by one of a frangible attachment and a combination of an adhesive attachment and a frangible attachment between the flap and the edge portion of said pouch.

24. The feminine hygiene kit according to claim 4, wherein said closure means is selected from an adhesive panel that sealingly covers said pouch opening and a closure flap that covers said pouch opening, and
wherein said sealed package containing said towelette is adhesively secured to an interior surface of said adhesive panel or said closure flap.

25. The feminine hygiene kit according to claim 24, wherein as said adhesive panel or closure flap is opened, said package containing said towelette is simultaneously extracted from said pouch and presented for ready access by the user.
